(19) United States
(12) Patent Application Publication
(10) Pub. No.: US 2008/0132520 A1
(43) Pub. Date: Jun. 5, 2008

(54) COMPOSITIONS, KITS AND METHODS FOR ADMINISTERING A TITRATION SCHEDULE COMPRISING BIFEPRUNOX COMPOUNDS

(76) Inventors: Antje A. Winsemius, Weesp (NL); Pieter W.A.J. Van Den Broeck, Weesp (NL); Luigi M. Barbato, Alpharetta, GA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/847,197

(22) Filed: Aug. 29, 2007

Related U.S. Application Data
(60) Provisional application No. 60/841,244, filed on Aug. 31, 2006.
(60) Provisional application No. 60/841,495, filed on Sep. 1, 2006.

(51) Int. Cl. A61K 31/497; A61P 25/18; C07D 413/10

(52) U.S. Cl. 514/254.02; 544/368

(57) ABSTRACT

The present disclosure is directed to a composition regimen, a titration kit, and methods of administration to facilitate the initiation of the treatment of at least one central nervous system condition or disorder by administering a plurality of dosage units of at least one bifeprunox compound, such as 7-[4-([1,1′-biphenyl]-3-ylmethyl)-1-piperazinyl ]-2(3H)-benzoxazolone (INN bifeprunox), according to a titration schedule.

This application claims priority to U.S. Provisional Application No. 60/841,244, filed Aug. 31, 2006, and U.S. Provisional Application No. 60/841 495, filed on Sep. 1, 2006, both of which are incorporated herein by reference in their entirety.

The present invention relates to compositions, kits, and methods for a titration schedule to facilitate the initiation of treatment of at least one central nervous system (CNS) condition or disorder by administering a plurality of dosage units of a composition comprising a compound, 7-[4-([1,1′-biphenyl]-3-ylmethyl)-1-piperazinyl]-2(3H)-benzoxazolone monomethane-sulfonate (INN bifeprunox).

The compound 7-[4-([1,1′-biphenyl]-3-ylmethyl)-1-piperazinyl]-2(3H)-benzoxa-zolone (INN bifeprunox) has the following formula:

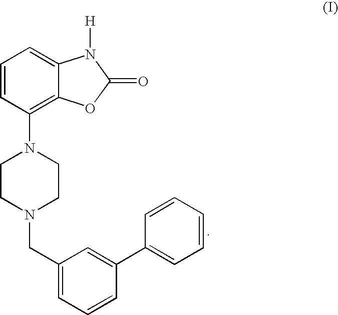

The hydrochloric acid salt of the above-referenced formula, i.e., (7-[4-([1,1′-biphenyl]-3-ylmethyl)-1-piperazinyl]-2(3H)-benzoxazolone, is described and claimed in International Publication No. WO 97/36893, and the monomethanesulfonate salt is described and claimed in International Publication No. WO 02/066449, which are incorporated herein by reference. In addition, International Publication No. WO 05/016898 describes and claims the different polymorphic forms of bifeprunox mesylate, which is incorporated herein by reference. And, the N-oxide of bifeprunox is described in International Publication No. WO 2007/023141, which is also incorporated herein by reference.

Bifeprunox (earlier known as DU 127090) binds to dopamine D₂-like receptors and 5-HT_{1A} receptors; it is a partial agonist at dopamine D_{2/3} receptors and also a partial agonist at serotonin 5-HT_{1A} receptors. See, e.g., Feenstra et al., International Publication No. WO 97/36893; Van Viet et al., DU 127090: A Highly Potent, Atypical Dopamine Receptor Ligand, 10(3) Journal of the European College of Neuropsychopharmacology (ECNP) S294 (2000); Feenstra et al., New 1-aryl-4-(Biarylmethylene)piperazines as Potential Atypical Anti-psychotic Agents with Mixed Dopamine D₂-Receptor- and Serotonin 5-HT_{1A} Receptor Affinities, 11 Bioorg. Med. Chem. Lett. 2345-2349 (2001); Feenstra et al., New Approaches in Antipsychotics: Design and Synthesis of Ligands Binding to Dopamine-D₂ and Serotonin 5-HT_{1A} Receptors, Clinical Candidates DU 127090 and SLV313, Drugs of the Future 27 (Suppl. A) (2002); Hesselink et al., DU 127090, SLV308 and SLV318: Characterization of a Chemically Related Class of Partial Dopamine Agonists with Varying Degrees of 5-HT1A Agonism, 10 Eur. J. Neurol. 2151 (2003).

Bifeprunox's affinity for both the dopamine D₂ and serotonin 5-HT_{1A} receptors makes it useful for the treatment of schizophrenia and other psychotic disorders. For example, bifeprunox can be of value for the treatment of conditions or disorders of the CNS caused by disturbances in either the dopamine or serotinergic systems, such as Parkinson's disease, aggression, anxiety disorders, autism, vertigo, depression, bipolar disorder, disturbances of cognition or memory, and further for example, schizophrenia and other psychotic disorders.

During clinical investigations on bifeprunox mesylate, the compound was well tolerated, however, undesirable adverse events such as nausea, vomiting and orthostatic hypertension can occur during the initiation of treatment, which may in some instances lead to early discontinuance of the treatment and/or noncompliance with the treatment plan. As such, there is a need to not only reduce these undesired side effects at the initiation of treatment, but also ensure compliance with the treatment plan. It is not uncommon in the treatment of patients with CNS disorders, in particular in the treatment of patients with antipsychotics, to start treatment with a titration period in which the dosage of the drug is gradually increased over a certain period of time. The length of the titration period, the dosages used, and the increments thereof, are, however, drug-dependent. Therefore, there is a need to find a suitable titration schedule for safe and effective initiation of the treatment with bifeprunox. It has now been surprisingly discovered that the tolerability to the compound can be improved and the occurrence of at least one of the undesired side effects can be reduced or prevented by gradual increase of the bifeprunox compound dose according to the titration schedule of this invention.

Accordingly, the present invention is directed to compositions, kits, and methods for administering a titration schedule to facilitate the initiation of treatment of at least one CNS condition or disorder by administering a plurality of dosage units comprising at least one bifeprunox compound, such as 7-[4-([1,1′-biphenyl]-3-ylmethyl)-1-piperazinyl]-2(3H)-benzoxazolone monomethane-sulfonate (INN bifeprunox). For example, the present invention comprises a composition regimen for titrating dosages of the at least one bifeprunox compound over a period of time up to a maintenance dosage for the treatment of at least one central nervous system condition or disorder, the composition regimen comprising a plurality of unit dosages according to a titration schedule, wherein each of the unit dosages comprises at least one bifeprunox compound, wherein the titration schedule comprises at least six time segments, and wherein the dosage of the at least one bifeprunox compound over the titration schedule increases to achieve a maintenance dose.

Further disclosed herein is a titration kit, comprising a plurality of unit dosages according to a titration schedule, wherein each of the unit dosages comprises at least one bifeprunox compound, and wherein the unit dosages increase in amount strengths over the titration period to achieve a maintenance dose.

In addition, the present invention is related to a method for initiating the treatment of at least one central nervous system condition or disorder in a subject in need thereof, comprising administering to the subject a composition regimen comprising a plurality of unit dosages according to a titration schedule, wherein each of the unit dosages comprises at least one bifeprunox compound, and wherein the unit dosages over the titration schedule increase in amount of the at least one bifeprunox compound to achieve a maintenance dose. Finally, disclosed herein is a method for reducing at least one side effect associated with the initiation of a bifeprunox treatment comprising administering a composition regimen comprising a plurality of unit dosages according to a titration schedule, wherein each of the unit dosages comprises at least one bifeprunox compound, and wherein the unit dosages over the titration schedule increase in amount of the at least one bifeprunox compound to achieve a maintenance dose.

Bifeprunox compounds are indicated for the treatment of CNS disorders including schizophrenia, other psychotic disorders and Parkinson's disease. In the framework of the present disclosure dosage strength is expressed in an amount equivalent to the amount of bifeprunox base. As used herein, the term “bifeprunox base” refers to the compound 7-[4-([1,1′-biphenyl]-3-ylmethyl)-1-piperazinyl]-2(3H)-benzoxa-zolone (INN bifeprunox) having the following formula:

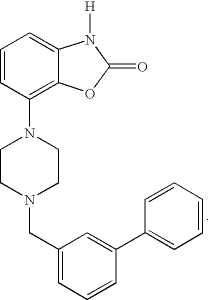

### The typical dosing regimen ranges from amounts equivalent to 0.05 mg to 60 mg bifeprunox base; doses may vary based in part on the severity of the CNS condition or disorder, as well as other conditions of the patient.

For example, a titration schedule dose or a maintenance dose for bifeprunox compounds can be a dose equivalent to 10 mg/day, 20 mg/day, 30 mg/day or 40 mg/day of bifeprunox base. In clinical studies, the efficacy, safety and tolerability profile of bifeprunox suggests it may be an effective agent to employ as a treatment option for patients with schizophrenia. Despite the efficacy, safety and tolerability profiles, bifeprunox treatment can result in adverse events during initiation of treatment, which may lead to the discontinuation of bifeprunox treatment and/or inconsistent use of the treatment.

As used herein, the term “bifeprunox compound(s)” refers to the active compound 7-[4-([1,1′-biphenyl]-3-ylmethyl)-1-piperazinyl]-2(3H)-benzoxa-zolone, its N-oxide and pharmaceutically acceptable salts, solvates and hydrates thereof and solvates and hydrates of the salts. When the N-oxide is used as the bifeprunox compound, the amount in milligrams is the same amount as the amount the person skilled in the art would select for the bifeprunox compound without the oxide. In addition, pharmaceutically acceptable salts of bifeprunox or its N-oxide may be obtained using standard procedures well known in the art, for example, by mixing a compound of the present invention with a suitable acid, for instance an inorganic acid or an organic acid.

In an embodiment of the present disclosure, the at least one bifeprunox compound comprises bifeprunox mesylate. For example, the at least one bifeprunox compound is bifeprunox mesylate. The bifeprunox mesylate may be chosen from the α, γ, or δ crystalline polymorphic forms, and mixtures thereof. For example, the at least one bifeprunox compound comprises at least one polymorphic form chosen from the α and γ polymorphic forms.

The crystalline polymorphic form of a bifeprunox mesylate according to the present disclosure is defined by at least the physicochemical parameters as disclosed in International Publication No. WO 2005/016898.

As used herein, the term “titration schedule” refers to a regimen of dosages of a pharmaceutically active agent over a period of time, based in part on a target dose and/or a maintenance dose. In at least one embodiment, the target dose and/or maintenance dose can be a dose equivalent to at least 10 mg/day, at least 20 mg/day, at least 30 mg/day or at least 40 mg/day of bifeprunox base and, for example, 10 mg/day, 20 mg/day, 30 mg/day or 40 mg/day of bifeprunox base. In particular, a target dose is 20 mg/day of at least one bifeprunox compound. In another embodiment, the target dose is 30 mg/day of at least one bifeprunox compound.

For example, the bifeprunox compound administered in the form of a plurality of unit dosages of a composition can be over the course of a titration schedule, wherein each time segment is one day, chosen from 6 to 14 consecutive days, such as from 6 to 10 days, and further, for example, from 6 to 8 days, and, in particular seven days. Alternatively, the titration schedule or treatment period can be divided into time segments other than one day, such as two, three or four day segments, or any time segment up to, but not including, seven days. In that case, the same unit dosage of bifeprunox compound can be administered every day within the time segment or a time segment may be a period of time where no unit dosage is administered, e.g., dose interruption or short-term non-compliance, allowing for the resumption of schedule compliance (i.e., dose or time segment) without the need to repeat any preceding time segment or dosing. The administration of the same unit dosage may thus be once daily administration of the full dosage, but also, could be, for example, twice daily administration of half of the dosage. The treatment period over which the titration schedule spans may be in part due to the lowest dose at which the titration starts, the amount of increase considered clinically acceptable, the target and/or maintenance dose; for example, a titration schedule may span over a larger number of days (such as 14 days) or it may span over a shorter period of time (such as 6 or 7 days).

Therefore, an embodiment of the present invention is a titration schedule wherein the number of time segments is at least six and with a titration period of at least six, twelve or eighteen days, respectively. Another embodiment relates to a titration schedule wherein the number of time segments is at least seven and with a titration period of at least seven, fourteen or twenty one days, respectively. The strength or amount of bifeprunox compound in each unit dosage increases incrementally over the subsequent titration schedule until a target dose and/or maintenance dose is reached. Alternatively, consecutive strengths or amounts of unit dosages may be given during the course of the titration such that over the entire titration schedule, an overall increase in strength or amount of the unit dosage results. For example, comparing the first unit dose to the last unit dose before the maintenance unit dosage is administered, there is an increase in the amount of bifeprunox compound being administered.

In one embodiment, each of the unit dosages of the composition are chosen from single strength doses equivalent to bifeprunox base from about 0.05 mg to about 0.07 mg, from about 0.07 mg to about 0.18 mg, from about 0.18 mg to about 0.35 mg, from about 0.35 mg to about 0.70 mg, from about 0.70 mg to about 1.4 mg, from about 1.4 mg to about 3.5 mg, from about 3.5 mg to about 7.0 mg, from about 7 mg to about 14 mg, from about 14 mg to about 28 mg, and from about 28 mg to about 32 mg. When choosing the different strengths of each unit dosage, the strength can be subsequent strengths within the group indicated above.

In a further embodiment, each of the unit dosages of the compositions are chosen from about 0.0550 mg to about 0.0675 mg, from about 0.10 mg to about 0.15 mg, from about 0.20 mg to about 0.30 mg, from about 0.4 mg to about 0.6 mg, from about 0.8 mg to about 1.2 mg, from about 1.5 mg to about 2.5 mg, from about 4.0 mg to about 6.0 mg, from about 8 mg to about 12 mg, from about 15 mg to about 25 mg, and from about 28 mg to about 30 mg of a bifeprunox compound. When choosing the different strengths of each unit dosage, the strength can be subsequent strengths within the group indicated above.

In yet a further embodiment of the titration schedule, the strength or amount of each unit dosage may be chosen from single strength doses equivalent to bifeprunox base about 0.0625 mg, 0.125 mg, about 0.25 mg, about 0.5 mg, about 1.0 mg, about 2.0 mg, about 5.0 mg, about 10 mg, about 20 mg, and about 30 mg.

Within the titration schedule, the unit dosages may increase in amount or strength of the at least one bifeprunox compound ranging from about 1.5 to 3 times that of the preceding dose and further for example, from about 2 to 2.5 times that of the preceding dose. In the case of the first dose of the titration schedule, there is no preceding dose, but with the remaining dosages of the titration schedule, a preceding dosage is available for consideration.

The composition regimen provided in the present disclosure can be in the form of a kit comprising, e.g., a plurality of unit dosages in the form of tablets for the titration schedule to arrive at the target and/or maintenance dose. The present invention is further directed to a kit to facilitate the treatment of at least one central nervous system condition or disorder comprising a plurality of unit dosages according to a titration schedule, each of the unit dosages comprise at least one bifeprunox compound, wherein the unit dosages over the titration schedule increase in an amount of the bifeprunox compound to achieve a maintenance dose. An embodiment, therefore, relates to a titration kit, comprising at least six sequential unit dosages, each of the unit dosages comprising at least one bifeprunox compound, wherein the unit dosages are of increasing strengths over the titration period. The strengths or amounts of each unit dosage can be chosen from the groups of different strengths indicated above. In another embodiment, the titration period of the kit is divided in at least six time segments (as defined previously).

The kit as described above can be in the form of a package, such as a blister package, the package comprising a plurality of tablets, e.g., each tablet having a different dose than another tablet and further for example, having indicia disposed adjacent to the tablets for displaying successive strengths and/or successive days.

In further embodiments, the kit can be in the form a package comprising a plurality of capsules, granular aerosols, suppositories and/or suspensions to form each unit dosage. Such dosage forms can be prepared by mixing, individually or together, the polymorphic forms of the bifeprunox compound (e.g., α, γ and/or δ of bifeprunox mesylate) with inert pharmaceutically acceptable excipients, carriers and/or pharmaceutically acceptable ingredients.

In the preparation of the compositions of the present disclosure, the active ingredients, i.e., at least one bifeprunox compound, may be mixed with solid, powdered ingredients, such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules, pressed into tablets, and/or any other known pharmaceutical form such as suppositories and/or suspensions.

Soft gelatin capsules may further be prepared containing a composition comprising a mixture of the active ingredients of the invention, vegetable oil, fat, or other suitable vehicle for soft gelatin capsules. Hard gelatin capsules may contain granules of the active ingredients. Hard gelatin capsules may also contain the active ingredients in combination with solid powdered ingredients such as lactose, saccharose, sorbitol, mannitol, potato starch, corn starch, amylopectin, cellulose derivatives or gelatin.

In addition, compositions of the present disclosure can comprise at least one pharmaceutical excipient. Non-limiting examples of suitable excipients include suspending agents (for example, gums, xanthans, cellulosics and sugars), humectants (for example, sorbitol), solubilizers (for example, ethanol, water, PEG and propylene glycol), surfactants (for example, sodium lauryl sulfate, Spans, Tweens, and cetyl pyridine), preservatives, antioxidants (for example, parabens, and vitamins E and C), anti-caking agents, coating agents, chelating agents (for example, EDTA), stabilizers, antimicrobial agents, antifungal or antibacterial agents (for example, parabens, chlorobutanol, phenol, sorbic acid), isotonic agents (for example, sugar, sodium chloride), thickening agents (for example, methyl cellulose), flavoring agents (for example, chocolate, thalmantin, aspartame, root beer or watermelon or other flavorings stable at pH 7 to 9), anti-foaming agents (e.g., simethicone, Mylicon®), disintegrants, flow aids, lubricants, adjuvants, colorants, diluents, moistening agents, preservatives, carriers, binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials), glidants and water insoluble or water soluble lubricants or lubricating agents.

The active ingredients may be separately premixed with the other non-active ingredients, before being mixed to form a formulation. The active ingredients may also be mixed with each other, before being mixed with the non-active ingredients to form a formulation.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

### EXAMPLES

### Example 1

### Materials and Methods

The alpha polymorphic form of bifeprunox mesylate was prepared as described in International Publication No. WO2005/016898. Sodium starch glycolate sodium stearyl fumarate, microcrystalline cellulose and lactose monohydrate were used in pharmaceutical grades available from common commercial sources.

### Example 2

### Preparation of a 10 mg Tablet

Tablets with a strength of 10 mg were prepared according to the following procedures (required quantities for all strengths are given in the Table below):

The microcrystalline cellulose was added to bin blender A and mixed for five (5) minutes to distribute. The material was transferred to bin blender B and mixed for five (5) minutes to distribute. The following ingredients were added to bin blender B; half (½) portion of lactose monohydrate bifeprunox mesylate, sodium starch glycolate and remaining half (½) portion of lactose monohydrate and mixed for twenty-five (25) to thirty-five (35) minutes.

The granulation was milled/de-agglomerated with a rotating impeller-screening mill using a conical screen equipped with a 0.6 mm screen or 0.8 mm screen. The granulation was transferred to a bin blender A. To bin blender A, sodium stearyl fumarate and colloidal silicon dioxide (for 20 mg only) was added and sieved through a #20 mesh. The granulation was mixed for a target of ten (10) to twenty-five (25) minutes, at 6 to 8 rpm.

The granulation was compressed on the rotary tablet press and collected in suitable containers.

The core tablets were loaded into the perforated coating pan.

Sufficient Opadry II film coating suspension was added to obtain about 3.5% of the core weight. The tablets were unloaded into appropriate containers.

| Ingredient | Amount (kg) per batch of 150 kg | | | | | | |
|---|---|---|---|---|---|---|---|
| Theoretical Yield | 1,250,000 | 1,000,000 | 833,333 | 833,333 | 714,285 | 625,000 | 555,556 |
| (tablet strength) | (0.25 mg) | (0.5 mg) | (1.0 mg) | (2.0 mg) | (5.0 mg) | (10 mg) | (20 mg) |
| Bifeprunox | 0.390 | 0.625 | 1.04 | 2.08 | 4.46 | 7.80 | 13.9 |
| Mesylate | | | | | | | |
| Lactose | 128.0 | 128.2 | 127.6 | 126.8 | 124.6 | 121.8 | 116.0 |
| Monohydrate | | | | | | | |
| Microcrystalline | 18.6 | 18.1 | 18.3 | 18.2 | 17.9 | 17.4 | 16.6 |
| Cellulose | | | | | | | |
| Sodium Starch | 0.750 | 0.750 | 0.750 | 0.750 | 0.750 | 0.750 | 0.750 |
| Glycolate | | | | | | | |
| Sodium Stearyl | 2.25 | 2.25 | 2.25 | 2.25 | 2.25 | 2.25 | 2.25 |
| Fumarate | | | | | | | |
| Colloidal Silicon | — | — | — | — | — | — | 0.59 |
| Dioxide | | | | | | | |
| Opadry II HP | 7.88 | — | — | — | 7.93 | — | — |
| White 85F18422 | | | | | | | |
| Opadry II HP Pink | — | 7.95 | — | — | — | — | — |
| 85F24345 | | | | | | | |
| Opadry II HP | — | — | 7.88 | — | — | — | — |
| Yellow 85F22185 | | | | | | | |
| Opadry II Blue | — | — | — | 7.88 | — | — | 7.92 |
| 85F20635 | | | | | | | |
| Opadry II Beige | — | — | — | — | — | 7.88 | — |
| 85F27126 | | | | | | | |
| Purified Water | 44.7 | 45.0 | 44.7 | 44.7 | 44.9 | 44.7 | 44.9 |

Tablets with a strength of 0.25 mg/tablet, 0.5 mg/tablet, 1.0 mg/tablet, 2.0 mg/tablet, 5.0 mg/tablet and 20 mg/tablet were prepared in a corresponding manner by decreasing or increasing the amount of bifeprunox mesylate, and compensating with the amount of lactose monohydrate and/or microcrystalline cellulose to come to the same final weight, with the understanding that a different coating component was used in order to obtain a different color for every tablet strength (see Table 1).

### Example 3

### Clinical Study

This study represented a randomized, double-blinded, placebo-controlled, sequential panel, rapid titration study of the safety and tolerability of bifeprunox compounds in subjects with schizophrenia. In particular, this study assessed the safety, tolerability and efficacy after a rapid titration of bifeprunox to a therapeutic dose of 40 mg/day in schizophrenia and schizoaffective subjects. Eligible subjects were hospitalized at the start of the screening period and entered a single-blind placebo run-in period (day -7 to -1) to assure that the 7-day antipsychotic drug-free criterion was met prior to bifeprunox titration. Subjects then began rapid titration at an initial bifeprunox starting dose of 0.25 mg with a high proposed therapeutic dose of 40 mg/day. Two titration schedules were evaluated in sequential order, with the titration period lasting 6 to 7 days depending on the titration schedule (Day 1 to Day 6 or 7).

Each listed dose in the titration schedule was administered once daily (QD) followed by the next higher dose the following day. If a subject did not tolerate a given dose, the subject was allowed to repeat that same dose for one extra day before increasing the dose to the next level. Only one repeat dose, however, was allowed during the entire titration period. Upon reaching the high proposed dose of 40 mg/day, the subject entered a 3-day maintenance period for this dose. Final assessments were performed on the day after the last maintenance period dose and follow-up assessments were performed 3 days after the last dose of study treatment.

Efficacy assessments (i.e., Positive and Negative Symptoms of Schizophrenia (PANSS) and Clinical Global Impression-Severity (CGI-S) were performed at screening, Day-1 and at the final assessment; Clinical Global Impressions-improvement (CGI-I was performed at the final assessment only. Safety was monitored throughout the study by assessment of physical examination, vital signs, adverse events (AEs), concomitant medications, clinical laboratory assessments, 12-lead electrocardiograms (ECGs), Simpson-Angus Scale (SAS) score and Barnes Akathisia Scale (BAS) score. The treatment phase lasted approximately two weeks, including follow-up assessment. Subjects remained hospitalized until the post-treatment follow-up assessment was completed or until their condition had clinically stabilized.

The study planned to include 20 subjects (10 subjects per panel). Twenty-seven subjects were screened and 18 were randomized (7 to bifeprunox and 1 to placebo in titration schedule 1 and 8 to bifeprunox and 2 to placebo titration schedule 2c). Ten subjects per panel were initially available as planned. Two subjects, however, dropped out of the first panel before the start of the study. As such, the study proceeded with the eighteen subjects.

Males and females between 18-55 years old meeting Diagnostic and Statistical Manual of Mental Disorders, 4^{th} edition, Text Revised (DSM-IV TR) diagnosis of Schizophrenia disorder (295.10, 295.20, 295.30, 295.60 and 295.90) or Schizoaffevtive disorder (295.70).

Bifeprunox were tablets taken orally at a total daily dose of 0.25 mg to 40 mg in the following titration schedules:

| Titration | Day | | | | | | |
|---|---|---|---|---|---|---|---|
| Schedule | 1 | 2 | 3 | 4 | 5 | 6 | 7 |
| 1 | 0.25 mg | 1 mg | 5 mg | 10 mg | 20 mg | 40 mg | N/A |
| 2c | 0.25 mg | 0.5 mg | 2 mg | 5 mg | 10 mg | 20 mg | 40 |
| | | | | | | | mg |

Placebo tablets matching the bifeprunox tablets were taken orally using the same dosing regimen as the test product.

Efficacy Results: Due to short duration of the treatment period used in this study, there was little benefit in bifeprunox treatment over the placebo in terms of improving the severity of symptoms of schizophrenia as measured by PANSS total scores, CGI-S and CGI-I scores and the responder rate based on the PANSS total score.

Safety Results: The temporal profile of intolerability observed during Titration Schedule 1 led the principal investigator to recommend a less aggressive titration schedule for the second panel of subjects. Other titration schedules, i.e., 2a, 2b and 2d (with jumps in dose from 0.25 mg to 2 mg to 10 mg, and 0.25 mg to 1 mg, respectively), were not optimal based on tolerance data. Titration schedule 2c, a longer and more conservative titration scheme, was therefore selected for the second panel of subjects. The choice of titration schedule 2c appeared to reduce the AEs observed during titration schedule 1, indicating an advantage with a more gradual increase from 0.25 mg to 10 mg and then up to 40 mg doses using a 7-day rather than 6-day titration schedule.

All subjects in the biteprunox group in titration schedule 1 reported at least one treatment of emergent adverse event (TEAE). Adverse events were most commonly reported between 0.25 mg to 10 mg dose levels, particularly at the 5 mg dose. The most commonly observed TEAEs were nervous system disorders (somnolence, dizziness, headache NOS and gait abnormal NOS), and gastrointestinal disorders (nausea and vomiting). Orthostatic hypotension, however, was also reported at the 1, 5, 10, and 40 mg dose levels. No TEAEs occurred at the 20 mg dose level. All subjects receiving bifeprunox in titration schedule 1 were considered to have at least 1 TEAE which was related to study treatment; all TEAEs occurring in the nervous system were considered to be related to study treatment. Three subjects receiving bifeprunox in titration schedule 1 reported TEAEs which were considered to be severe, with the most commonly reported events occuring in the nervous system. Severe TEAEs of orthostatic hypotension, nausea, vomiting NOS, and rigors were reported. All severe TEAEs occurred at the 1, 5 and 10 dose levels.

Two subjects receiving bifeprunox in titration schedule 1 had vital sign abnormalities on the same day as relevant AEs (either dizziness or orthostatic hypotension). Changes in blood pressure and heart rate were associated with TEAEs of dizziness and orthostatic hypotension which led to rechallenge of a dose level and/or to discontinuation of subjects from the study, with TEAEs of headache BOS, nausea and somnolence also commonly associated with rechallenge and/or discontinuation. Three subjects (1 male and both female subjects) experienced TEAEs which lead to discontinuation of the study treatment, with all subject reporting TEAEs which were related to the study treatment and 2 of the 3 subject having required rechallenge of a dose level. Rechallenge was successful for both of these subjects, who proceed with titration to higher doses, however, they were eventually discontinued die to further TEAEs.

All subjects in the bifeprunox group in titration schedule 2c reported at least one TEAE. Adverse events were reported at all dose levels, although few events were reported at the 10 mg dose. As more patients were titrated up to the 40 mg dose than in titration schedule 1, more AEs were reported at the 40 mg dose level in titration schedule 2c. The most commonly observed TEAEs were nervous system disorders (dizziness, headache NOS and somnolence) and gastrointestinal disorders (dyspepsia and nausea). Six out of 8 subjects receiving bifeprunox in titration schedule 2c were considered to have TEAEs which were related to the study treatment, with the most commonly reported events occurring in the nervous system. In contrast to titration schedule 1, fewer subjects reported events of somnolence which were considered to be related to the study treatment, which no TEAEs of anorexia, gait abnormal NOS or orthostatic hypotension were reported. Three subjects receiving bifeprunox in titration schedule 2c reported TEAEs which were considered to be severe. All three subjects reported severe TEAEs of dizziness and a severe TEAE of pallor was reported in one subject. All severe TEAEs occurred at the 2 mg and 5 mg dose level. One subject, receiving placebo in titration schedule 2c, reported an serious adverse event (SAE) (face injury).

Four subjects receiving bifeprunox in titration schedule 2c had vital sign abnormalities same days as AEs of dizziness. Changes in blood pressure and heart rate were associated with TEAEs of dizziness which led to rechallenge of a dose level and/or to discontinuation of subjects from the study. One female subject experienced a TEAE of dizziness (related to the study treatment) which led to discontinuation of the study treatment, and had previously required rechallenge of a dose level. Two additional subjects required re-challenge of a dose level and subsequently completed the titration schedule up to the maximum dose of 40 mg. Overall, however, more subjects with vital sign abnormalities (both as AEs and as markedly abnormal results) in titration schedule 2c completed titration up to the 40 mg dose level than in titrations schedule 1.

Neither titration schedule of bifeprunox appeared to have any effects on BAS, SAS, ECG or clinical laboratory evaluations. None of the changes noted in physical examination were judged to be clinically meaningful in either schedule.

Subjects in titration schedule 1 received bifeprunox at doses of 0.25, 1, 5, 10, 20, and 40 mg over 6 consecutive days. In this titration schedule, most AEs occurred in the 0.25 to 10 mg dose range. Once the 10 mg dose was reached, daily increases to 20 to 40 mg were well-tolerated. Titration schedule 2c had a slower titration up to the 10 mg level with subjects dosed at 0.25, 0.5, 2, 5, 10, 20, and 40 mg bifeprunox over 7 consecutive days. This longer and more conservative titration schedule resulted in fewer withdrawals from the study due to intolerable effects and appeared to reduce the AEs observed during titration schedule 1. Bifeprunox exposure was approximately 1.3 to 1.5 times higher in titration schedule 2c than titration schedule 1 on the final study day, while exposure was comparable between the two schedules for the metabolites. The results of this study generally indicated that titration of bifeprunox from 0.25 mg to 40 mg over 7 days was better tolerated than titration over a 6-day period. It, however, should be noted that the final, maintenance dose of this study was 40 mg and the titration schedule, i.e., 6 to 7 days, was based in part on the level of this final, maintenance dose.

What is claimed:

1. A composition regimen to facilitate the initiation of treatment of at least one central nervous system condition or disorder comprising a plurality of unit dosages according to a titration schedule, wherein each of the unit dosages comprises at least one bifeprunox compound, and wherein the unit dosages over the titration schedule increase in amount of the at least one bifeprunox compound to achieve a maintenance dose.

2. The composition regimen according to claim 1, wherein the titration schedule spans at least six time segments.

3. The composition regimen according to claim 2, wherein the titration schedule spans at least seven time segments.

4. The composition regimen according to claim 2 or 3, wherein each of the time segments is chosen, independent of one another, from one day, two days, three days, and four days.

5. The composition regimen according to claim 1, wherein each of the unit dosages is chosen from single strength doses ranging from about 0.05 mg to about 0.07 mg, from about 0.07 mg to about 0.18 mg, from about 0.18 mg to about 0.35 mg, from about 0.35 mg to about 0.70 mg, from about 0.70 mg to about 1.4 mg, from about 1.4 to about 3.5 mg, from about 3.5 to about 7.0 mg, from about 7.0 to about 14.0 mg, from about 14.0 to about 28.0 mg, and from about 28.0 mg to about 32.0 mg.

6. The composition regimen according to claim 5, wherein each of the unit dosages is chosen from single strength doses ranging from about 0.0550 mg to about 0.0675 mg, from about 0.10 mg to about 0.15 mg, from about 0.20 mg to about 0.30 mg, from about 0.4 mg to about 0.6 mg, from about 0.8 mg to about 1.2 mg, from about 1.5 mg to about 2.5 mg, from about 4.0 to about 6.0 mg, from about 8.0 mg to about 12.0 mg, from about 15.0 mg to about 25.0 mg, and from about 28.0 mg to about 30.0 mg.

7. The composition regimen according to claim 6, wherein each of the unit dosages is chosen from single strength doses of 0.0625 mg, 0.125 mg, 0.25 mg, 0.5 mg, 1.0 mg, 2.0 mg, 5.0 mg, 10 mg, 20 mg, and 30 mg.

8. The composition regimen according to claim 1, wherein the unit dosages of the at least one bifeprunox compound increase over the titration schedule in an amount ranging from about 1.5 to 3 times of the amount of the preceding unit dosage.

9. The composition regimen according to claim 8, wherein the unit dosages of the at least one bifeprunox compound increase over the titration schedule in an amount ranging from 2 to 2.5 times of the amount of the preceding unit dosage.

10. The composition regimen according to claim 1, wherein the bifeprunox compound comprises bifeprunox mesylate.

11. The composition regimen according to claim 10, wherein the bifeprunox compound comprises an alpha polymorph of bifeprunox mesylate.

12. The composition regimen according to claim 1, wherein the maintenance dose is 20 mg/day of the at least one bifeprunox compound.

13. The composition regimen according to claim 1, wherein the maintenance dose is 30 mg/day of the at least one bifeprunox compound.

15. The composition regimen according to claim 1, wherein the at least one central nervous system disorder comprises schizophrenia.

14. A titration kit comprising a plurality of unit dosages according to a titration schedule, wherein each of the unit dosages comprises at least one bifeprunox compound, and wherein the unit dosages over the titration schedule increase in an amount of the at least one bifeprunox compound to achieve a maintenance dose.

15. The kit according to claim 14, wherein the titration schedule spans at least six time segments.

16. The kit according to claim 15, wherein the titration schedule spans at least seven time segments.

17. The kit according to claims 15 or 16, wherein each of the time segments is chosen, independent of one another, from one day, two days, three days, and four days.

18. The kit according to claim 14, wherein each of the unit dosages is chosen from single strength doses from about 0.05 mg to about 0.07 mg, from about 0.07 mg to about 0.18 mg, from about 0.18 mg to about 0.35 mg, from about 0.35 mg to about 0.70 mg, from about 0.70 mg to about 1.4 mg, from about 1.4 to about 3.5 mg, from about 3.5 to about 7.0 mg, from about 7.0 to about 14.0 mg, from about 14.0 to about 28.0 mg, and from about 28.0 mg to about 32.0 mg.

19. The kit according to claim 18, wherein each of the unit dosages is chosen from single strength doses from about 0.0550 mg to about 0.0675 mg, from about 0.10 mg to about 0.15 mg, from about 0.20 mg to about 0.30 mg, from about 0.4 mg to about 0.6 mg, from about 0.8 mg to about 1.2 mg, from about 1.5 mg to about 2.5 mg, from about 4.0 to about 6.0 mg, from about 8.0 mg to about 12.0 mg, from about 15.0 mg to about 25.0 mg, and from about 28.0 mg to about 30.0 mg.

20. The kit according to claim 19, wherein each of the unit dosages is chosen from single strength doses of 0.0625 mg, 0.125 mg, 0.25 mg, 0.5 mg, 1.0 mg, 2.0 mg, 10 mg, 20 mg, and 30 mg.

21. The kit according to claim 14, wherein the unit dosages of the at least one bifeprunox compound increase over the titration schedule in an amount ranging from about 1.5 to 3 times of the amount of preceding unit dosage.

22. The kit according to claim 21, wherein the unit dosages of the at least one bifeprunox compound increase over the titration schedule in an amount ranging from 2 to 2.5 times of the amount of the preceding unit dosage.

23. The kit according to claim 14, wherein the bifeprunox compound comprises bifeprunox mesylate.

24. The kit according to claim 23, wherein the bifeprunox compound comprises an alpha polymorph of bifeprunox mesylate.

25. A method for administering a titration schedule to facilitate the initiation of treatment of at least one central nervous system condition or disorder in a subject in need thereof, comprising:
administering to the subject a composition regimen comprising a plurality of unit dosages according to the titration schedule, wherein each of the unit dosages comprises at least one bifeprunox compound, and wherein the unit dosages over the titration schedule increase in amount of the at least one bifeprunox compound to achieve a maintenance dose.

26. The method according to claim 25, wherein the titration schedule spans at least six time segments.

27. The method according to claim 25, wherein the titration schedule spans at least seven time segments.

28. The method according to claim 26 or 27, wherein each of the time segments is chosen, independent of one another, from one day, two days, three days, and four days.

29. The method according to claim 25, wherein the unit dosages of the at least one bifeprunox compound increase over the titration schedule in an amount ranging from about 1.5 to 3 times of the amount of the preceding unit dosage.

30. The method according to claim 29, wherein the unit dosages of the at least one bifeprunox compound increase over the titration schedule in an amount ranging from 2 to 2.5 times of the amount of the preceding unit dosage.

31. The method according to claim 25, wherein each of the unit dosages is chosen from single strength doses from about 0.05 mg to about 0.07 mg, from about 0.07 mg to about 0.18 mg, from about 0.18 mg to about 0.35 mg, from about 0.35 mg to about 0.70 mg, from about 0.70 mg to about 1.4 mg, from about 1.4 to about 3.5 mg, from about 3.5 to about 7.0 mg, from about 7.0 to about 14.0 mg, from about 14.0 to about 28.0 mg, and from about 28.0 mg to about 32.0 mg.

32. The method according to claim 31, wherein each of the unit dosages is chosen from single strength doses from about 0.0550 mg to about 0.0675 mg, from about 0.10 mg to about 0.15 mg, from about 0.20 mg to about 0.30 mg, from about 0.4 mg to about 0.6 mg, from about 0.8 mg to about 1.2 mg, from about 1.5 mg to about 2.5 mg, from about 4.0 to about 6.0 mg, from about 8.0 mg to about 12.0 mg, from about 15.0 mg to about 25.0 mg, and from about 28.0 mg to about 30.0 mg.

33. The method according to claim 32, wherein each of the unit dosages is chosen from single strength doses of 0.0625 mg, 0.125 mg, 0.25 mg, 0.5 mg, 1.0 mg, 2.0 mg, 10 mg, 20 mg, and 30 mg.

34. The method according to claim 25, wherein the bifeprunox compound comprises bifeprunox mesylate.

35. The method according to claim 34, wherein the bifeprunox compound comprises an alpha polymorph of bifeprunox mesylate.

36. A method for reducing at least one side effect associated with initiating treatment with at least one bifeprunox compound comprising:
administering a composition regimen comprising a plurality of unit dosages according to a titration schedule, wherein each of the unit dosages comprises at least one bifeprunox compound, and wherein the unit dosages over the titration schedule increase in amount of the at least one bifeprunox compound to achieve a maintenance dose.

37. The method according to claim 36, wherein the titration schedule spans at least six time segments.

38. The method according to claim 37, wherein the titration schedule spans at least seven time segments.

39. The method according to claim 37 or 38, wherein each of the time segments is chosen, independent of one another, from one day, two days, three days, and four days.

40. The method according to claim 36, wherein the unit dosages of the at least one bifeprunox compound increase over the titration schedule in an amount ranging from about 1.5 to 3 times of the amount of the preceding unit dosage.

41. The method according to claim 40, wherein the unit dosages of the at least one bifeprunox compound increase over the titration schedule in an amount ranging from 2 to 2.5 times of the amount of the preceding unit dosage.

42. The method according to claim 36, wherein each of the unit dosages is chosen from single strength doses from about 0.05 mg to about 0.07 mg, from about 0.07 mg to about 0.18 mg, from about 0.18 mg to about 0.35 mg, from about 0.35 mg to about 0.70 mg, from about 0.70 mg to about 1.4 mg, from about 1.4 to about 3.5 mg, from about 3.5 to about 7.0 mg, from about 7.0 to about 14.0 mg, from about 14.0 to about 28.0 mg, and from about 28.0 mg to about 32.0 mg.

43. The method according to claim 42, wherein each of the unit dosages is chosen from single strength doses from about 0.0550 mg to about 0.0675 mg, from about 0.10 mg to about 0.15 mg, from about 0.20 mg to about 0.30 mg, from about 0.4 mg to about 0.6 mg, from about 0.8 mg to about 1.2 mg, from about 1.5 mg to about 2.5 mg, from about 4.0 to about 6.0 mg, from about 8.0 mg to about 12.0 mg, from about 15.0 mg to about 25.0 mg, and from about 28.0 mg to about 30.0 mg.

44. The method according to claim 43, wherein each of the unit dosages is chosen from single strength doses of 0.0625 mg, 0.125 mg, 0.25 mg, 0.5 mg, 1.0 mg, 2.0 mg, 10 mg, 20 mg, and 30 mg.

45. The method according to claim 36, wherein the bifeprunox compound comprises bifeprunox mesylate.

46. The method according to claim 45, wherein the bifeprunox compound comprises an alpha polymorph of bifeprunox mesylate.

47. A method for treating a patient suffering from schizophrenia, comprising:
initiating treatment with a composition regimen comprising a plurality of unit dosages of a composition according to a titration schedule, wherein each of the unit dosages comprises at least one bifeprunox compound, and wherein the unit dosages over the titration schedule increase in amount of the at least one bifeprunox compound; and
maintaining treatment with administration of a maintenance dose of the at least one bifeprunox compound.

48. The method according to claim 47, wherein the maintenance dose is 20 mg/day of the at least one bifeprunox compound.

49. The method according to claim 47, wherein the maintenance dose is 30 mg/day of the at least one bifeprunox compound.

50. A method for treating a patient suffering from schizophrenia, comprising:
initiating treatment with a composition regimen comprising a plurality of unit dosages according to a titration schedule, wherein each of the unit dosages comprises at least one bifeprunox compound, and wherein the unit dosages over the titration schedule increase in amount of the at least one bifeprunox compound, wherein the titration schedule spans at least six time segments, and wherein each of the unit dosages is chosen from single strength doses ranging from about 0.05 mg to about 0.07 mg, from about 0.07 mg to about 0.18 mg, from about 0.18 mg to about 0.35 mg, from about 0.35 mg to about 0.70 mg, from about 0.70 mg to about 1.4 mg, from about 1.4 to about 3,5 mg, from about 3.5 to about 7.0 mg, from about 7.0 to about 14.0 mg, from about 14.0 to about 28.0 mg, and from about 28.0 mg to about 32.0 mg; and
maintaining treatment with administration of a maintenance dose of the at least one bifeprunox compound.

51. The method according to claim 50, wherein the maintenance dose is 20 mg/day of the at least one bifeprunox compound.

52. The method according to claim 50, wherein the maintenance dose is 30 mg/day of the at least one bifeprunox compound.

53. A method for treating a patient suffering from schizophrenia, comprising:
initiating treatment with a composition regimen comprising a plurality of unit dosages according to a titration schedule, wherein each of the unit dosages comprises at least one bifeprunox compound, wherein the unit dosages over the titration schedule increase in amount of the at least one bifeprunox compound, and wherein the initiation of treatment diminishes at least one side effect associated with administration of the at least one bifeprunox compound without the composition regimen; and
maintaining treatment with administration of a maintenance dose of the at least one bifeprunox compound.

54. The method according to claim 53, wherein the maintenance dose is 20 mg/day of the at least one bifeprunox compound.

55. The method according to claim 53, wherein the maintenance dose is 30 mg/day of the at least one bifeprunox compound.

56. A method for treating a patient suffering from schizophrenia, comprising: initiating treatment with a composition regimen comprising a plurality of unit dosages according to a titration schedule, wherein each of the unit dosages comprise at least one bifeprunox compound, wherein the unit dosages titrate the at least one bifeprunox compound from 0.25 mg per day to a maintenance dose over at least six days; and
maintaining treatment with the maintenance dose.

57. The method according to claim 56, wherein the maintenance dose is 20 mg/day of the at least one bifeprunox compound.

58. The method according to claim 56, wherein the maintenance dose is 30 mg/day of the at least one bifeprunox compound.

59. A method for treating a patient suffering from schizophrenia, comprising:
initiating treatment with at least one bifeprunox compound by dosing the patient according to the following composition regimen: Day 1, 0.25-mg of bifeprunox compound per day; Day 2, 0.5-mg of bifeprunox compound per day; Day 3, 1.0-mg of bifeprunox compound per day; Day 4, 2.0-mg of bifeprunox compound per day; Day 5, 5-mg of bifeprunox compound per day; Day 6, 1 0-mg of bifeprunox compound per day; Day 7, 20-mg of biteprunox compound per day; and
maintaining treatment by administering a maintenance dose of bifeprunox compound per day thereafter.
